(19) United States
(12) Patent Application Publication
(10) Pub. No.: US 2022/0263598 A1
(43) Pub. Date: Aug. 18, 2022

(54) ENDOSCOPE SYSTEM, PROCESSING DEVICE, AND CONTROL METHOD OF SIGNAL TRANSMISSION

(71) Applicant: OLYMPUS CORPORATION, Tokyo (JP)

(72) Inventor: Norihito YAMADA, Tokyo (JP)

(73) Assignee: OLYMPUS CORPORATION, Tokyo (JP)

(21) Appl. No.: 17/735,454

(22) Filed: May 3, 2022

Related U.S. Application Data
(63) Continuation of application No. PCT/JP2019/044201, filed on Nov. 11, 2019.

(51) Int. Cl. H04L 1/00; H04N 17/00; A61B 1/00

(52) U.S. Cl. CPC H04L 1/0006; H04L 1/0028; A61B 1/00114; A61B 1/00009; H04N 17/00

(57) ABSTRACT

An endoscope system includes: an endoscope configured to generate an image signal; a processing device configured to process the image signal; a plurality of signal lines configured to transmit a signal between the endoscope and the processing device; and a processor configured to detect a communication error of the signal lines, and determine a communication protocol to allocate a signal to be transmitted by the respective signal lines according to a detection result of the communication error, the determination of the communication protocol including determining whether to transmit a signal in two ways based on number of signal lines in which the communication error is not detected.

### CROSS-REFERENCE TO RELATED APPLICATION

This application is a continuation of International Application No. PCT/JP2019/044201, filed on Nov. 11, 2019, the entire contents of which are incorporated herein by reference.

### BACKGROUND

### 1. Technical Field

The present disclosure relates to an endoscope system, a processing device, and a control method of signal transmission.

### 2. Related Art

In the medical field, endoscope systems have been used to observe the inside of a body of a subject (for example, JP-A-2017-153769). Generally, an endoscope has a flexible insertion portion in a long thin shape to be inserted into the inside of the body of a subject, such as patient, and illuminates the inside of the body of the subject with illumination light emitted from a distal end of this insertion portion. The illumination light is supplied to the insertion portion by a light source device. In the endoscope, reflected light of the illumination light is received by an imaging unit at the distal end of the insertion portion, to capture an in-vivo image. The in-vivo image captured by the imaging unit of the endoscope is subjected to predetermined image processing in a processing device of an endoscope system, and then displayed on a display of the endoscope system. A user, such as a doctor, observes an organ of the subject based on the in-vivo image displayed on the display.

### SUMMARY

In some embodiments, an endoscope system includes: an endoscope configured to generate an image signal; a processing device configured to process the image signal; a plurality of signal lines configured to transmit a signal between the endoscope and the processing device; and a processor configured to detect a communication error of the signal lines, and determine a communication protocol to allocate a signal to be transmitted by the respective signal lines according to a detection result of the communication error, the determination of the communication protocol including determining whether to transmit a signal in two ways based on number of signal lines in which the communication error is not detected.

In some embodiments, provided is a processing device transmitting a signal by using a plurality of signal lines between the processing device and an endoscope. The processing device includes: a processor configured to detect a communication error of the signal lines, and determine a communication protocol to allocate a signal to be transmitted by the respective signal lines according to a detection result of the communication error, the determination of the communication protocol including determining whether to transmit a signal in two ways based on number of signal lines in which the communication error is not detected.

In some embodiments, provided is a control method of signal transmission by using a plurality of signal lines between an endoscope and a processing device. The method includes: detecting a communication error of the signal lines; and determining a communication protocol to allocate a signal to be transmitted by the respective signal lines according to a detection result of the communication error, the determining the communication protocol including determining whether to transmit a signal in two ways based on number of signal lines in which the communication error is not detected.

In some embodiments, provided is a processing device transmitting a signal by using a plurality of signal lines between the processing device and an endoscope. The processing device includes: a processor configured to detect a communication error of the signal lines, and to cause a single signal line to transmit a signal in two ways when number of signal lines in which the communication error is not detected is equal to or fewer than number of types of signals essential to be transmitted.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of an endoscope system according to one embodiment of the disclosure;

FIG. 2 is a block diagram illustrating a schematic configuration of the endoscope system according to one embodiment of the disclosure;

FIG. 3 is a diagram explaining about communications between an endoscope and a processing device in the endoscope system according to one embodiment of the disclosure;

FIG. 4 is a flowchart explaining communication control processing that is performed by the endoscope system according to one embodiment of the disclosure;

FIG. 5 is a diagram illustrating a change example (Part 1) of a communication protocol;

FIG. 6 is a diagram illustrating a change example (Part 2) of the communication protocol;

FIG. 7 is a diagram illustrating an example of a selection screen of a priority mode in data transmission;

FIG. 8 is a diagram illustrating a change example (Part 3) of the communication protocol;

FIG. 9A is a diagram explaining about a detection example of a communication error between the endoscope and the processing device in the endoscope system according to one embodiment of the disclosure;

FIG. 9B is a diagram explaining about a detection example of a communication error between the endoscope and the processing device in the endoscope system according to one embodiment of the disclosure;

FIG. 10 is a diagram illustrating an example of output of change information of the communication protocol; and

FIG. 11 is a diagram illustrating an example of a configuration of data including meta data.

### DETAILED DESCRIPTION

Hereinafter, a form (hereinafter, “embodiment”) to implement the disclosure will be explained. In an embodiment, an endoscope system for medical use that captures and displays an image of the inside of the body of a subject, such as patient, will be explained as one example of a system implementing a communication control method according to the disclosure. Moreover, this embodiment is not intended to limit this disclosure. Furthermore, it will be explained using like reference symbols for like parts in descriptions in the drawings.

### Embodiment

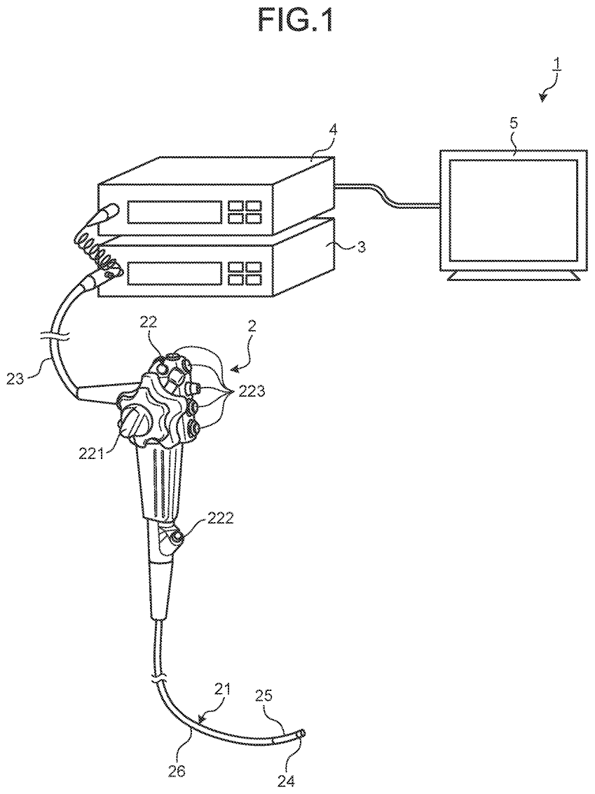

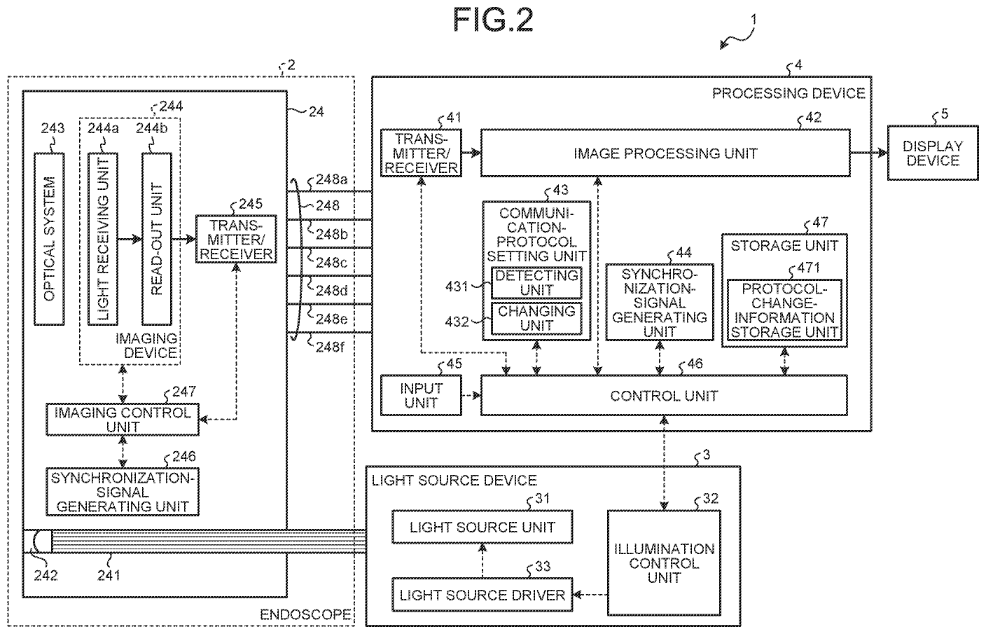

FIG. 1 is a diagram illustrating a schematic configuration of an endoscope system according to one embodiment of the disclosure. FIG. 2 is a block diagram illustrating a schematic configuration of the endoscope system according to the embodiment.

An endoscope system 1 illustrated in FIG. 1 and FIG. 2 includes an endoscope 2 that captures an in-vivo image of a subject by inserting a distal end portion into the body of the subject, a light source device 3 that generates illumination light to be emitted from a distal end of the endoscope 2, a processing device 4 that subjects an image signal captured by the endoscope 2 to predetermined signal processing, and that controls overall operations of the endoscope system 1, and a display device 5 that displays an in-vivo image generated by the signal processing of the processing device 4. In FIG. 2, transmission of a signal relating to image data is indicated by a solid line, and transmission of a signal relating to control is indicated by a broken arrow.

The endoscope 2 includes an insertion portion 21 that is flexible and has a long thin shape, an operation portion 22 that is connected to a proximal end of the insertion portion 21, and that accepts an input of various kinds of operation signals, and a universal cord 23 that extends to a direction different from a direction in which the insertion portion 21 extends from the operation portion 22, and that contains various kinds of cables to be connected to the light source device 3 and the processing device 4.

The insertion portion 21 includes a distal end portion 24 having an imaging device 244 in which pixels generating a signal by receiving light and by performing photoelectric conversion are arranged two-dimensionally, a bendable portion 25 that is constituted of multiple bending pieces to be bendable, and a flexible tube portion 26 that is connected to a proximal end side of the bendable portion 25, and that has a long flexible shape. The insertion portion 21 is inserted into a body cavity of the subject, and captures an image of a subject, such as a living tissue at a position at which outside light does not reach, with the imaging device 244.

The distal end portion 24 includes a light guide 241 that is constituted of plural glass fibers or the like, and that forms a light guide path for light emitted by the light source device 3, an illumination lens 242 that is arranged at a distal end of the light guide 241, an optical system 243 for condensation of light, the imaging device 244 that is arranged at an image forming position of the optical system 243, and that receives light condensed by the optical system 243 to photoelectric-convert into an electric signal, and subjects it to predetermined signal processing, a transmitter/receiver 245 that transmits and receives a signal between itself and the processing device 4, a synchronization-signal generating unit 246 that generates a clock signal (synchronization signal) to be a reference of the operation of the endoscope 2, and an imaging control unit 247 that controls the imaging device 244 and the transmitter/receiver 245.

The optical system 243 is constituted of one or more pieces of lenses, and has an optical zooming function to change an angle of view and a focusing function to change a focal point.

The imaging device 244 generates an electric signal (image signal) by subjecting light from the optical system 243 to photoelectric conversion. Specifically, the imaging device 244 includes a light receiving unit 244a in which plural pixels, each of which has a photodiode to accumulate electric charges according to an amount of light, a capacitor that converts the electric charge transferred from the photodiode to a voltage level, and the like are arranged in a matrix form, and each of the pixels photoelectric-converts light from the optical system 243, to generate an electric signal, and a read-out unit 244b that sequentially reads the electric signal generated by a pixel arbitrarily set as a subject to be read out of the pixels of the light receiving unit 244a, to output as an image signal. The imaging device 244 is implemented by using, for example, a charge coupled device (CCD) image sensor, or a complementary metal oxide semiconductor (CMOS) image sensor.

The transmitter/receiver 245 transmits the image signal output by the imaging device 244 to the processing device 4, and receives a control signal transmitted from the processing device 4, to transmit to the imaging control unit 247.

The synchronization-signal generating unit 246 generates a clock signal (synchronization signal) to be a reference of the operation of the endoscope 2 (the distal end portion 24), and outputs the generated synchronization signal to the processing device 4. The synchronization signal passes through the imaging control unit 247 and the transceiver/receiver 245, and is then transmitted to the processing device 4. The synchronization signal generated by the synchronization-signal generating unit 246 includes a horizontal synchronization signal and a vertical synchronization signal.

The imaging control unit 247 performs a driving control of the respective components including the imaging device 244, an input/output control of information with respect to the respective components, and the like. The imaging control unit 247 controls respective parts of the distal end portion 24 based on a control signal transmitted from the processing device 4. The imaging control unit 247 is constituted of a general-purpose processor, such as central processing unit (CPU), a dedicated processor as respective arithmetic circuits performing specific functions, such as application specific integrated circuit (ASIC), or the like.

The endoscope 2 has a memory (not illustrated) that stores an execution program for the imaging device 244 to perform various kinds of operations, and data including identification information of the endoscope 2. The identification information includes unique information (ID) of the endoscope 2, a model year, specification information, transmission mode, and the like. Moreover, the memory may temporarily store image data generated by the imaging device 244 and the like.

The operation portion 22 includes a bending knob 221 to bend the bendable portion 25 in an up-and-down direction and a left-and-right direction, a treatment-instrument insertion portion 222 that inserts a treatment instrument, such as biopsy forceps, electrosurgical knife, and inspection probe, into a body cavity of the subject, and plural switches 223 that are an operation input unit to input an operation instructing signal of peripheral devices, such as air feeder, water feeder, screen display control, in addition to the processing device 4. The treatment instrument inserted from the treatment-tool insertion portion 222 is exposed from an opening (not illustrated) through a treatment instrument channel (not illustrated) in the distal end portion 24.

The universal cord 23 incorporates at least the light guide 241, and a bundled cable 248 in which plural signal lines are bundled. In the present embodiment, the bundled cable 248 has four signal lines (a first signal line 248a, a second signal line 248b, a third signal line 248c, and a fourth signal line 248d), a power source cable 248e to supply power to the endoscope 2 from the processing device 4, and a ground cable 248f to stabilize a ground potential of the endoscope 2. These four signal lines are allocated as signal lines that transmit or receive an image signal generated by the imaging device 244, a driving signal (control signal) to drive the imaging device 244, a synchronization signal generated in the processing device 4, and a synchronization signal generated in the distal end portion 24 (endoscope 2). In the present embodiment, an example in which a transmission capacity of these four signal lines are the same will be explained, but the transmission capacities of the respective signal lines may be partially different, or be different from one another. In the present embodiment, it will be explained, assuming that an electrical signal is transmitted by using the signal line, but an optical signal may be transmitted.

Subsequently, a configuration of the light source device 3 will be explained. The light source device 3 includes a light source unit 31, an illumination control unit 32, and a light source driver 33.

The light source unit 31 is constituted of a light source that emits illumination light by using plural lenses and the like, and emits illumination light including light of a predetermine wavelength band by driving of the respective light sources. The configuration of the light source unit 31 will be described later. The light source unit 31 may be configured to have a single light source that emits illumination light, or may be configured to have plural light sources that generate illumination light by light of wavelength bands different from one another.

The illumination control unit 32 controls an amount of power to be supplied to the respective light sources based on the control signal (luminance control signal) from the control unit 46, and controls timing of driving the respective light sources included in the light source unit 31. The luminance control signal is, for example, a pulse signal forming a predetermined waveform.

The light source driver 33 supplies an electric current to the light source unit 31 under control of the illumination control unit 32, and thereby causes the respective light sources to emit light.

Next, a configuration of the processing device 4 will be explained. The processing device 4 includes a transmitter/receiver 41, an image processing unit 42, a communication-protocol setting unit 43, a synchronization-signal generating unit 44, an input unit 45, a control unit 46, and a storage unit 47.

Besides transmitting a control signal or a synchronization signal output by the control unit 46 to the endoscope 2 (distal end portion 24), the transmitter/receiver 41 transmits an image signal transmitted from the endoscope 2 to the image processing unit 42, and transmits a synchronization signal received from the endoscope 2 to the control unit 46.

The image processing unit 42 receives image data of illumination light of respective colors captured by the imaging device 244 from the endoscope 2. When an analog image signal is received from the endoscope 2, the image processing unit 42 generates a digital image signal by performing A/D conversion. Moreover, when an image signal is received as an optical signal from the endoscope 2, the image processing unit 42 generates a digital image signal by performing photoelectric conversion. The image processing unit 42 is constituted of a general-purpose processor, such as CPU, a dedicated processor as various kinds of arithmetic circuits performing specific functions, such as ASIC, or the like.

The image processing unit 42 generates an image signal for display by subjecting an image signal received from the endoscope 2 to predetermined image processing. The image processing unit 42 outputs the generated image signal for display to the display device 5. The predetermined image processing is synchronization processing, tone correction processing, color correction processing, and the like. Furthermore, the image processing unit 42 may be configured to include a frame memory that holds the generated image signal for display.

The communication-protocol setting unit 43 changes communication allocation of signal lines by changing a communication protocol according to whether communication is enabled in the respective signal lines in the bundled cable 248. The communication-protocol setting unit 43 is constituted of a general-purpose processor, such as CPU, a dedicated processor as various kinds of arithmetic circuits performing specific functions, such as ASIC. In the present embodiment, at least plural signal lines (a first signal line 248a to a fourth signal line 248d) and the communication-protocol setting unit 43 constitute a communication control system.

The communication-protocol setting unit 43 includes a detecting unit 431 that detects a communication error of a signal line, and a changing unit 432 that changes a communication protocol based on a detection result of the detecting unit 431. Details of processing performed by the detecting unit 431 and the changing unit 432 will be described later.

The synchronization-signal generating unit 44 generates a clock signal (synchronization signal) to be a reference of the operation of the processing device 4, and outputs the generated synchronization signal to the light source device 3, respective components of the processing device 4, and the endoscope 2. The synchronization signal generated by the synchronization-signal generating unit 44 includes a horizontal synchronization signal and a vertical synchronization signal, similarly to the synchronization-signal generating unit 246.

Therefore, the light source device 3, the image processing unit 42, the control unit 46, and the endoscope 2 operate in synchronization with one another based on the generated synchronization signal.

The input unit 45 is implemented by using a keyboard, a mouse, a switch, and a touch panel, and accepts an input of various kinds of signals, such as an operation instruction signal to instruct an operation of the endoscope system 1. The input unit 45 may include a switch arranged in the operation portion 22, or a portable terminal, such as an external tablet computer.

The control unit 46 performs a driving control of respective components including the endoscope 2 and the light source device 3, an input/output control of information with respect to the respective components, and the like. The control unit 46 refers to control information data for imaging control stored in the storage unit 47 (for example, read out timing, and the like), and transmits it as a control signal through a predetermined signal line included in the bundled cable 248 to the distal end portion 24. Moreover, the control unit 46 refers to control information data for light source control stored in the storage unit 47, and causes the illumination control unit 32 to control the light source unit. The control unit 46 is constituted of a general-purpose processor, such as CPU, or a dedicated processor as various kinds of arithmetic circuits, such as ASIC performing a specific function.

The storage unit 47 stores data including various kinds of programs to operate the endoscope system 1, and various kinds of parameters necessary for the operation of the endoscope system 1. Furthermore, the storage unit 47 stores identification information of the processing device 4. The identification information includes unique information (ID) of the processing device 4, a model year, specification information, and the like. Moreover, the storage unit 47 includes a protocol-change-information storage unit 471 that stores communication protocols according to whether communication is enabled in respective signal lines. The protocol-change-information storage unit 471 stores information relating to a communication protocol to which a signal type to be communicated is allocated according to whether communication is enabled in a signal line.

Moreover, the storage unit 47 stores various kinds of programs including a program for performing a communication control method of the processing device 4. The various kinds of programs can be widely distributed by recording on a computer-readable recording medium, such as a hard disk, a flash memory, a CD-ROM, a DVD-ROM, and a flexible disk. The various kinds of programs described above can also be acquired by downloading through a communication network. The communication network herein is implemented by, for example, an existing public line network, a local area network (LAN), a wide area network (WAN), or the like, and may be either wired or wireless communication network.

The storage unit 47 having the above configuration is implemented by using a read only memory (ROM) in which various kinds of programs and the like are installed in advance, a RAM or a hard disk that stores arithmetic parameters and data of respective processing, and the like.

In the present embodiment, an example in which the endoscope 2 operates based on a control signal from the processing device 4, regarding the processing device 4 as a master unit and the endoscope 2 as a slave unit will be explained.

The display device 5 displays an image corresponding to an image signal received from the processing device 4 (image processing unit 42) through a video cable. The display device 5 is constituted of a monitor, such as liquid crystal display and an organic electroluminescence (EL) display.

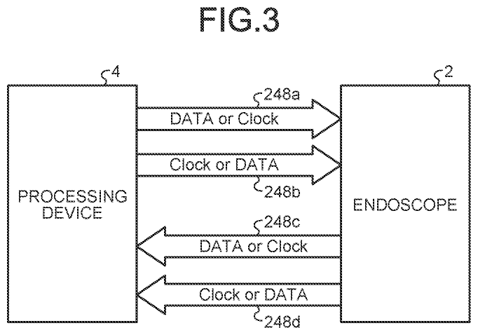

Next, communication between the endoscope 2 and the processing device 4 will be explained, referring to FIG. 3. FIG. 3 is a diagram explaining communication between an endoscope and a processing device in the endoscope system according to one embodiment of the disclosure. The endoscope 2 and the processing device 4 transmit and receive a signal between the devices by the first signal line 248a, the second signal line 248b, the third signal line 248c, and the fourth signal line 248d described above. The endoscope 2 and the processing device 4 perform communication of a signal in two ways by these signal lines. The respective signal lines transmit a signal of a communication type allocated to respective lines. “DATA” in FIG. 3 indicates an image signal or a control signal, and “CLOCK” indicates a synchronization signal. Specifically, by allocating respective signal types (DATA or CLOCK) to be transmitted to the respective signal lines, the signals are transmitted and received. FIG. 3 illustrates an example in which a function of transmitting a signal (the control signal and the synchronization signal) to the endoscope 2 from the processing device 4 is allocated to the first signal line 248a and the second signal line 248b, and a function of transmitting a signal (the image signal and the synchronization signal) to the processing device 4 from the endoscope 2 is allocated to the third signal line 248c and the fourth signal line 248d.

For the respective signals, a signal line capable of two-way communication is used. As communication that can be implemented by these signal lines, a clock synchronization mode, such as serial peripheral interface (SPI) communication for one-way communication and an inter-integrated circuit (I2C) communication for two-way communication, or an asynchronous communication mode, such as universal asynchronous receiver/transmitter (UART) communication can be adopted, but it is not limited thereto.

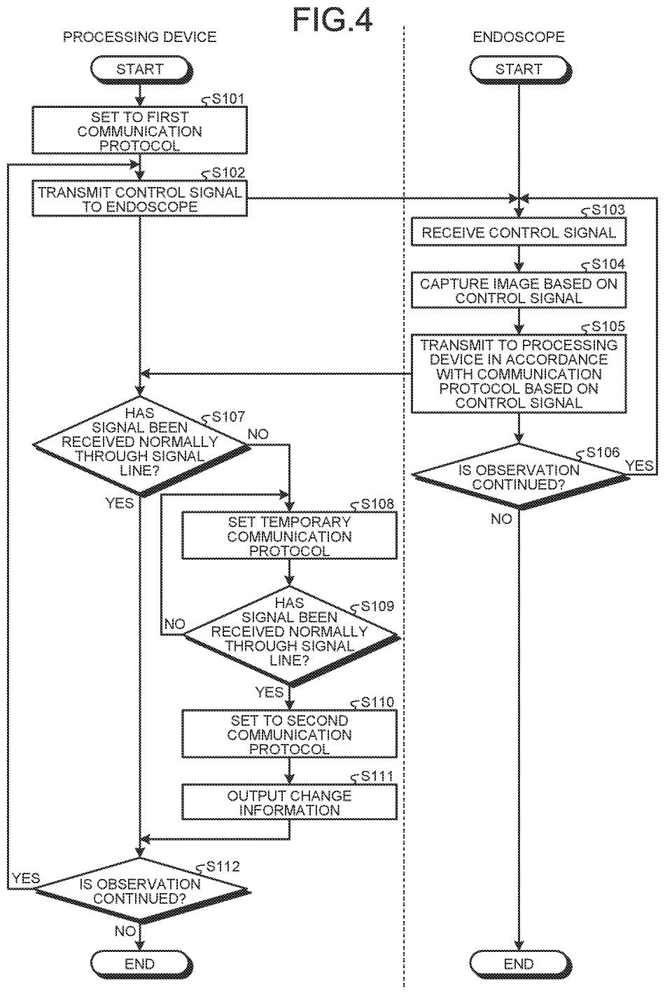

Subsequently, a communication control between the endoscope 2 and the processing device 4 will be explained, referring to FIG. 4 to FIG. 9B. FIG. 4 is a flowchart explaining communication control processing performed by the endoscope system according to one embodiment of the disclosure.

In the processing device 4, the communication-protocol setting unit 43 sets a communication protocol to a first communication protocol after activation of the device (step S101). The first communication protocol is a protocol in which all four signal lines are in a communication-enabled state, and to all of the signal lines, either one of transmission of an image signal, transmission of a control signal, transmission of a synchronization signal generated by the synchronization-signal generating unit 44, and transmission of a synchronization signal generated by the synchronization-signal generating unit 246 is allocated.

The processing device 4 transmits setting information of a communication protocol, or a control signal including control information, such as information relating to imaging, to the endoscope 2 (step S102).

The endoscope 2 receives a control signal from a processing layer t4 (step S103). The endoscope 2 performs imaging processing based on the received control signal (step S104).

Thereafter, the endoscope 2 transmits data according to the communication protocol based on the control information to the processing device 4 (step S105). At step S105, the endoscope 2 transmits predetermined data (ACK data or image data described later) by using a signal line corresponding to the set communication protocol.

After data transmission, the endoscope 2 determines whether to continue observation with the endoscope 2. The endoscope 2 determines whether to continue observation by checking an ON state of the power of itself, or an observation end instruction from the processing device 4. For example, when determining that the power is in the ON state (step S106: YES), the endoscope 2 shifts to step S103. On the other hand, for example when the power of the endoscope 2 is turned OFF (step S106: NO), this communication control processing is ended.

Meanwhile, the communication-protocol setting unit 43 determines whether a signal has been received normally through a signal line to perform reception in communication of the respective signal lines (step S107). In the processing device 4, the detecting unit 431 determines whether a signal to be received (herein, an image signal and a synchronization signal generated by the synchronization-signal generating unit 246) has been received. In contrast, in the endoscope 2, the imaging control unit 247 determines whether a signal to be received (herein, a control signal and a synchronization signal generated by the synchronization-signal generating unit 44) has been received. Communication confirmation of a signal in the endoscope 2 is performed by reception of predetermined data (ACK data) from a device of a reception side. When predetermined data (ACK data) is not received from the processing device 4, the imaging control unit 247 transmits a signal indicating that the signal has not been received. In this case, the imaging control unit 247 transmits, for example, a change command of the communication protocol. For communication confirmation, a publicly-known error detection method can be applied. The detecting unit 431 detects whether the endoscope 2 can receive a signal based on a presence or absence of the change command from the endoscope 2.

At step S107, when it is determined that all signals have been received normally based on the detection result of the detecting unit 431 (step S107: YES), the communication-protocol setting unit 43 shifts to step S112. In contrast, when it is determined that at least one signal has not been received normally (step S107: NO), the communication-protocol setting unit 43 shifts to step S108.

At step S108, the changing unit 432 refers to the protocol-change-information storage unit 471, and changes to a temporary communication protocol using a normal signal line. When a usable signal line is not available at this time, the processing is ended.

At step S109 subsequent to step S108, the detecting unit 431 determines whether a signal has been received normally through a signal line to perform reception in the temporary communication protocol. At step S105, when it is determined that all signals have been received normally based on the detection result of the detecting unit 431 (step S109: YES) similarly to step S107, the communication-protocol setting unit 43 shifts to step S110. In contrast, when it is determined that at least one signal has not bee received normally (step S109: NO), the communication-protocol setting unit 43 returns to step S108, and re-sets a temporary communication protocol.

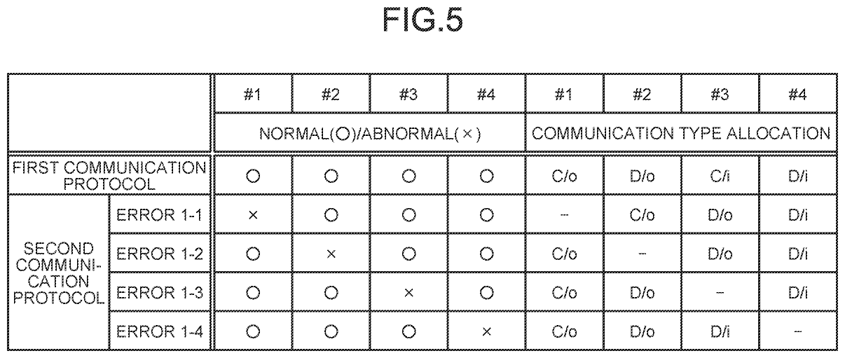

An example of a communication protocol change will be explained. FIG. 5 to FIG. 8 illustrate communication viewed from the processing device 4 side, and transmission of an image signal is indicated as D/i (input of an image signal), transmission of a control signal is indicated as D/o (output of a control signal), transmission of a synchronization signal generated by the synchronization-signal generating unit 44 is indicated as C/i (input of a synchronization signal), and transmission of a synchronization signal generated by the synchronization-signal generating unit 246 is indicated as C/o (output of a synchronization signal). FIG. 5 to FIG. 8 illustrate an example in which C/o is allocated to the first signal line 248a (#1), D/o is allocated to the second signal line 248b (#2), C/i is allocated to the third signal line 248c (#3), and D/i is allocated to the fourth signal line 248d (#4). Moreover, in the present embodiment, signals for which communication is essential are two signals, which are the control signal and the image signal.

FIG. 5 is a diagram illustrating a change example (Part 1) of the communication protocol. The change example illustrated in FIG. 5 is one example of the communication protocol when either one of the signal lines is in a communication-disabled state. When one signal line is in the communication-disabled state (Errors 1-1 to 1-4), that is, when communication is performed by using three signal lines, in a temporary communication protocol, D/i, D/o, and C/o are allocated to the three signal lines. For example, Error 1-1 is a state in which communication is disabled in the first signal line 248a, and as a temporary communication protocol, the C/o is allocated to the second signal line 248b, D/o is allocated to the third signal line 248c, and D/i is allocated to the fourth signal line 248d.

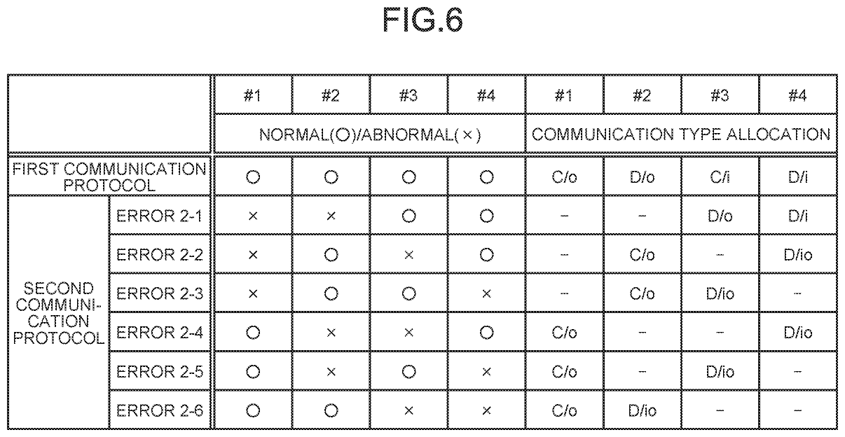

FIG. 6 is a diagram illustrating a change example (Part 2) of the communication protocol. The change example illustrated in FIG. 6 is one example of the communication protocol when two signal lines are in a communication-disabled state. When two signal lines are in a communication-disabled state (Errors 2-1 to 2-6), that is, when communication is performed by using two signal lines, in the second communication protocol, D/i and D/o, or C/o and D/io are allocated to the two signal lines. D/io is assigned to rolls of performing transmission of an image signal and transmission of a control signal in a single signal line. For example, Error 2-1 is a state in which communication is disabled in the first signal line 248a and the second signal line 248b, and as a temporary communication protocol, D/o is allocated to the third signal line 248c, and D/i is allocated to the fourth signal line 248d. Furthermore, Error 2-2 is a state in which communication is disabled in the first signal line 248a and the third signal line 248c, and as a temporary communication protocol, C/o is allocated to the second signal line 248b, and D/io is allocated to the fourth signal line 248d.

When two signal lines are in a communication-disabled state, priority may be given to allocating C/o and D/io to the two signal lines (Error 2-2 to 2-6). In this case, because D/io is assigned with rolls of performing transmission of an image signal and transmission of a control signal in a single signal line, the speed of data transmission decreases to some extent, but highly precise image transmission is possible because data communication can be performed in a clock synchronization mode.

Moreover, in a state in which communication is disabled in two signal lines, priority may be given to allocating D/i and D/o to two signal lines (Error 2-1). In this case, because D/i and D/o can be transmitted by respective communication lines, although an image quality decreases to some extent, data can be transmitted, keeping a frame rate.

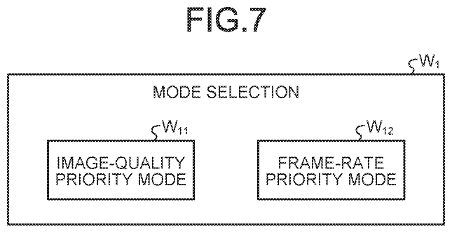

Furthermore, it may be configured such that a user can select a mode in which an image quality is prioritized (Error 2-2 to 2-6) or a mode in which a frame rate is prioritized (Error 2-1). Thus, allocation of signal suitable for a need of a user is possible. FIG. 7 is a diagram illustrating an example of a selection screen of a priority mode in data transmission. A user selects either one of a button for a mode in which the image quality is prioritized (image-quality-priority-mode selection button W₁₁) or a button for a mode in which the frame rate is prioritized (frame-rate-priority-mode selection button W₁₂) on a selection screen W₁ (refer to FIG. 7) displayed, for example, on the display device 5 to input it. The communication-protocol setting unit 43 selects a mode according to the input by the user.

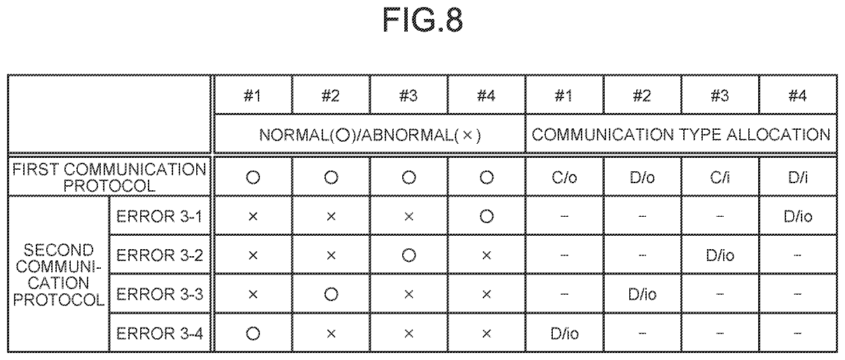

FIG. 8 is a diagram illustrating a change example (Part 3) of the communication protocol. The example illustrated in FIG. 8 is one example of the communication protocol when three signal lines are in a communication-disabled state. When three signal lines are in a communication-disabled state (Errors 3-1 to 3-4), that is, when communication is performed by using a single signal line, in a temporary communication protocol, D/io is allocated to the signal line enabled to communicate. For example, Error 3-1 is a state in which communication is disabled in the first signal line 248a, the second signal line 248b, and the third signal line 248c, and as a temporary communication protocol, D/io is allocated to the fourth signal line 248d.

As illustrated in FIG. 6 and FIG. 8, when the number of signal lines enabled to communicate is equal to or fewer than the number of essential signals to be communicated (in this example, two, the control signal and the image signal), a pattern in which a function of performing two-way communication is assigned to a single signal line occurs in a communication protocol.

The allocation patterns illustrated in FIG. 5, FIG. 6, and FIG. 8 are stored in the protocol-change-information storage unit 471.

Moreover, in a state in which communication is disabled in three signal lines, if signal lines are disabled to perform communication gradually (for example, one by one), it can be dealt with by changing the second communication protocol gradually. However, in a state in which three signals are disable to perform communication at the same time, a device on one side (in this example, the processing device 4) cannot determine that three signal lines are disabled to perform communication at the same time. In this case, the communication-protocol setting unit 43 first determines whether communication is possible as for a signal line allocated as a signal line for the processing device 4 to receive a signal, and sets a temporary communication protocol, and thereafter, determines whether communication is possible as for a signal line allocated again as a signal line for the processing device 4 to receive a signal (corresponding to step S109).

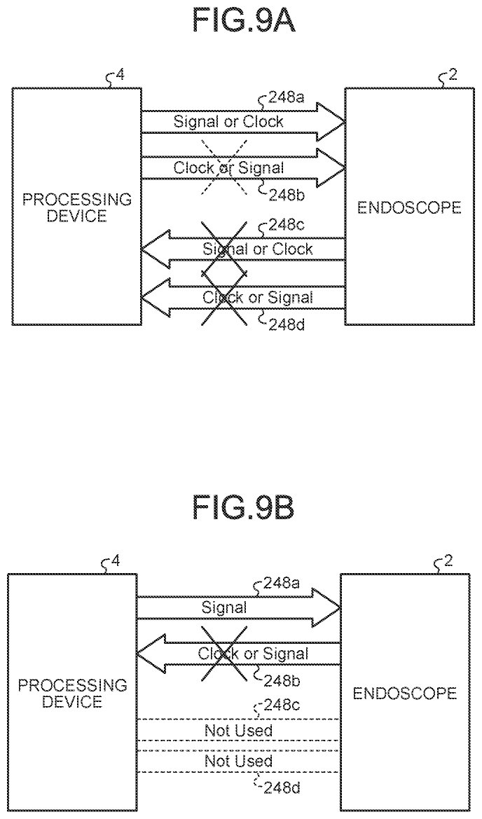

FIG. 9A and FIG. 9B are diagrams explaining about a detection example of a communication error between the endoscope and the processing device in the endoscope system according to one embodiment of the disclosure. FIG. 9A and FIG. 9B illustrate an example in which communication is disabled in the second signal line 248b to the fourth signal line 248d. Moreover, as the first communication protocol, the first signal line 248a and the second signal line 248b are allocated to signal lines for the processing device 4 to transmit a control signal and a synchronization signal to the endoscope 2, and the third signal line 248c and the fourth signal line 248d are allocated to signal lines for the endoscope 2 to transmit an image signal and a synchronization signal to the processing device 4 (refer to FIG. 9A).

The control unit 46 first transmits a control signal and a synchronization signal to the endoscope 2 through the first signal line 248a and the second signal line 248b, and receives a signal by a signal line of the reception side (in this example, the third signal line 248c and the fourth signal line 248d). The communication-protocol setting unit 43 determines that the third signal line 248c and the fourth signal line 248d are disabled to perform communication based on a detection result of the detecting unit 431. In this case, a change command from the endoscope 2 cannot be received either. When it is in a state in which the third signal line 248c and the fourth signal line 248d are disabled to perform communication, the changing unit 432 allocates types of signals to be transmitted and received to the first signal line 248a and the second signal line 248b as the second communication protocol. For example, the changing unit 432 causes the first signal line 248a to transmit a control signal to the endoscope 2, and sets a temporary communication protocol to receive an image signal from the endoscope 2 to the second signal line 248b. In this case, the third signal line 248c and the fourth signal line 248d are not used for communication.

The control unit 46 controls to transmit a control signal to the endoscope 2 through the first signal line 248a, and to receive an image signal from the endoscope 2 through the second signal line 248b in accordance with the settings by the communication-protocol setting unit 43. At this time, when it is determined that the second signal line 248b is disabled to perform communication (for example, step S105: NO) based on a reception state from the second signal line 248b, the communication-protocol setting unit 43 re-sets a temporary communication protocol (refer to FIG. 9B). The communication-protocol setting unit 43 re-sets to a temporary communication protocol in which a control signal is transmitted to the endoscope 2 and an image signal is received from the endoscope 2 to the first signal line 248a as the temporary communication protocol.

When a communication error occurs in any of the four signal lines, out of synchronization signals transmitted and received between the endoscope 2 and the processing device 4, transmission of a synchronization signal (synchronization signal generated by the synchronization-signal generating unit 246) transmitted from the endoscope 2 to the processing device 4 stops. In this case, if a synchronization signal generated in the processing device 4 (synchronization signal generated by the synchronization-signal generating unit 44) is transmitted to the endoscope 2 by communication, processing in the endoscope 2 is performed based on this synchronization signal. Meanwhile, for example, in Errors 3-1 to 3-4, transmission/reception of a synchronization signal itself stops, and in this case, devices are synchronized by an asynchronous communication or the like.

Referring back to FIG. 4, at step S110, the changing unit 432 sets the set temporary communication protocol to the second communication protocol. At this time, as described above, as for a communication error in plural signal lines at the same time, particularly, in a signal line to transmit a signal from the master side, appropriate communication allocation can be performed by repeating determination whether communication is possible one by one (steps S108, S109).

At step S111, the communication-protocol setting unit 43 outputs information relating to the changed communication protocol (change information). At this time, the change information is output to the display device 5 through the image processing unit 42. In the display device 5, information relating to the change information, for example, textural information is displayed, or information indicating the change (sound or light) is output. Thereafter, the communication-protocol setting unit 43 shifts to step S112.

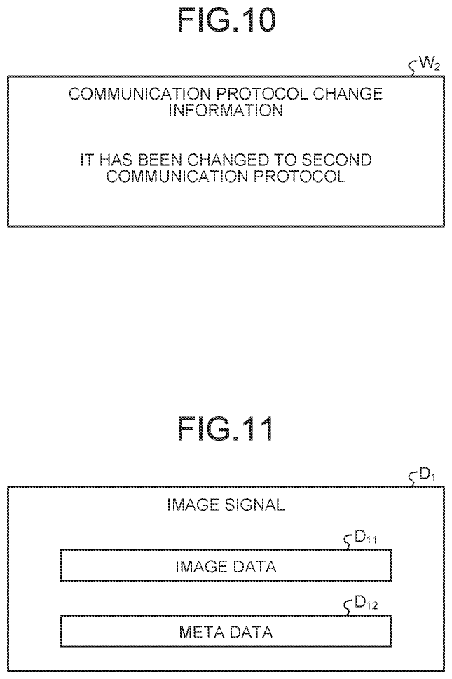

FIG. 10 is a diagram illustrating one example of output of the change information of the communication protocol. When a communication protocol is changed, the communication-protocol changing unit 43 causes the display device 5 to display, for example, change information W₂ illustrated in FIG. 10. By the display of the change information W₂ illustrated in FIG. 10, textural information indicating that the communication protocol has been changed to the second communication protocol is displayed.

The control unit 46 controls communication in accordance with the set communication protocol.

At step S112, the control unit 46 determines whether observation of the endoscope 2 is being continued. The control unit 46 determines whether the observation is continued, for example, by checking ON/OFF of the power of the endoscope. When it is determined, for example, that the power of the endoscope 2 is ON, and that the observation of the endoscope 2 is being continued (step S112: YES), the control unit 46 shifts to step S102. At this time, processing is continued by using the communication protocol set at step S104 or S105 instead of the first communication protocol. In contrast, when it is determined, for example, that the power of the endoscope 2 is OFF, and that the observation of the endoscope 2 is not continued (step S112: NO), the control unit 46 ends the processing. The communication control processing explained above is performed after the endoscope 2 and the processing device 4 start operating, and is performed while the endoscope 2 is operating.

In the embodiment explained above, when an error occurs in communication of signal lines in a system in which a control signal, an image signal, and a synchronization signal are transmitted and received by using plural signal lines to perform two-way communication between the endoscope 2 and the processing device 4, by allocating types of signals to be communication through a normal signal line, and communication of at least the control signal and the image signal are maintained. According to the present embodiment, because communication of a control signal and an image signal is maintained, even when an error occurs in communication between devices, by changing a communication protocol, communication for image output can be maintained.

In the embodiment described above, information indicating that imaging has been performed with the communication protocol set at step S108 may be generated as metadata, and may be stored in the storage unit 47. FIG. 11 is a diagram illustrating an example of a configuration of data including meta data. An image signal D₁ illustrated in FIG. 11 is constituted of image data D₁₁ to form an image, and meta data D₁₂ that includes information indicating that imaging has been performed with a communication protocol described above.

The embodiment to implement the disclosure has so far been explained, but the disclosure is not to be limited only to the embodiment described above. The disclosure can include various embodiments not described herein, and the like.

In the above embodiment, a case in which the processing device 4 is a master unit, and the endoscope 2 is a slave unit, and the endoscope 2 operates according to a control signal from the processing device 4 has been explained, but the master-slave relationship may be opposite. When the endoscope 2 is the master unit, the communication protocols in FIGS. 5 to 7 are to be processing viewed from the endoscope 2.

Moreover, as a configuration in which either one can be the master unit, it may be configured such that a device that has received a change command serves as a master, and changes the communication protocol.

Furthermore, in the embodiment described above, a case in which communication between the endoscope 2 and the processing device 4 is implemented by four signal lines has been explained, but not limited to four, for example, ten or more signal lines may be used.

Moreover, in the embodiment described above, an example in which respective signal lines are signal lines capable of performing two-way communication has been explained, but as long as an essential signal to be communicated can be transmitted and received, at least some signal lines may be signal lines of one-way communication.

Furthermore, in the embodiment described above, the light source device 3 has been explained to be configured separately from the endoscope 2, but it may be configured such that the light source device is arranged in the endoscope 2 by arranging a semiconductor laser at a distal end of the endoscope 2, or the like. Moreover, the function of the processing device 4 may be given to the endoscope 2.

Furthermore, in the embodiment described above, the light source device 3 has been explained to be configured separately from the processing device 4, but the light source device 3 and the processing device 4 may be integrated, and it may be configured such that the light source unit 31, the illumination control unit 32, and the light source driver 33 are arranged inside the processing device 4.

Moreover, in the embodiment described above, the endoscope system according to the disclosure has been explained as the endoscope system 1, an observation target of which is a living tissue inside the body of a subject and the like, and that uses the flexible endoscope 2, but it is also applicable to an endoscope system that uses a rigid endoscope, an endoscope for industrial use to observe characteristics of a material, a capsule endoscope, and an optical endoscope, such as fiberscope and an optical viewing tube, in which a camera head is connected to its eyepiece.

As above, the endoscope system, the computer-readable recording medium, the processing device, and the endoscope according to the disclosure are useful for maintaining image output even when an error occurs in communication between devices.

According to the disclosure, an effect that even when an error occurs in communication between devices, communication for image output can be maintained is produced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
an endoscope configured to generate an image signal;
a processing device configured to process the image signal;
a plurality of signal lines configured to transmit a signal between the endoscope and the processing device; and
a processor configured to
detect a communication error of the signal lines, and
determine a communication protocol to allocate a signal to be transmitted by the respective signal lines according to a detection result of the communication error, the determination of the communication protocol including determining whether to transmit a signal in two ways based on number of signal lines in which the communication error is not detected.

2. The endoscope system according to claim 1, wherein
the processor is configured to allocate a type of signal to be transmitted to a signal line in which the communication error is not detected based on the detection result of the communication error.

3. The endoscope system according to claim 2, wherein
the respective signal lines are capable of performing two-way transmission of a signal.

4. The endoscope system according to claim 3, wherein
the processor is configured to cause a single signal line to transmit a signal in two ways when number of signal lines in which the communication error is not detected is equal to or fewer than number of types of essential signal to be transmitted.

5. The endoscope system according to claim 1, wherein
the processor is configured to change to a communication protocol in which a synchronization signal generated in the processing device is transmitted in priority to a synchronization signal generated in the endoscope.

6. The endoscope system according to claim 5 wherein
the processor is configured to
detect the communication error of the signal lines based on a signal reception state in the processing device,
change the communication protocol according to a detection result of a signal line to perform a signal reception in the processing device, and
detect a communication error in a signal line in which the communication error has not been detected after the communication protocol is changed.

7. The endoscope system according to claim 1, wherein
the endoscope system is configured such that, in a state in which the communication error of the signal lines is not detected, to the signal lines, either one of the image signal generated by the endoscope, a control signal generated by the processing device, a synchronization signal generated by the endoscope, and a synchronization signal generated by the processing device is allocated.

8. The endoscope system according to claim 7, wherein
a communication protocol in a state in which the communication error of the signal lines is not detected is a first communication protocol, and
a communication protocol in a state in which the communication error of the signal lines is detected is a second communication protocol.

9. The endoscope system according to claim 8, wherein
the endoscope system is configured such that, in a state in which communication is performed by the second communication protocol, an image-quality priority mode in which an image quality is prioritized and a frame-rate priority mode in which a frame rate is prioritized can be switched therebetween.

10. The endoscope system according to claim 9, wherein
the signal lines are a first signal line, a second signal line, a third signal line, and a fourth signal line, and
the endoscope system is configured such that when the image-quality priority mode is selected in a state in which a communication error is detected in the first signal line and the second signal line, the image signal is transmitted by the third signal line and the control signal is transmitted by the fourth signal line, and when the frame-rate priority mode is selected in a state in which a communication error is detected in the first signal line and the second signal line, the image signal and the control signal are transmitted in two ways by the third signal line and the synchronization signal generated by the processing device is transmitted by the fourth signal line.

11. The endoscope system according to claim 7, wherein
the endoscope system is configured such that the image signal and the control signal are transmitted in two ways to a single signal line.

12. The endoscope system according to claim 7, wherein
the signal lines are a first signal line, a second signal line, a third signal line, and a fourth signal line, and
the endoscope system is configured such that, in a state in which a communication error is detected in the first signal line, the synchronization signal generated by the processing device is transmitted by the second signal line, the image signal is transmitted by the third signal line, and the control signal is transmitted by the fourth signal line.

13. A processing device transmitting a signal by using a plurality of signal lines between the processing device and an endoscope, the processing device comprising:
a processor configured to
detect a communication error of the signal lines, and
determine a communication protocol to allocate a signal to be transmitted by the respective signal lines according to a detection result of the communication error, the determination of the communication protocol including determining whether to transmit a signal in two ways based on number of signal lines in which the communication error is not detected.

14. A control method of signal transmission by using a plurality of signal lines between an endoscope and a processing device, the method comprising:
detecting a communication error of the signal lines; and
determining a communication protocol to allocate a signal to be transmitted by the respective signal lines according to a detection result of the communication error, the determining the communication protocol including determining whether to transmit a signal in two ways based on number of signal lines in which the communication error is not detected.

15. A processing device transmitting a signal by using a plurality of signal lines between the processing device and an endoscope, the processing device comprising:
a processor configured to
detect a communication error of the signal lines, and
to cause a single signal line to transmit a signal in two ways when number of signal lines in which the communication error is not detected is equal to or fewer than number of types of signals essential to be transmitted.
